(19) United States
(12) Patent Application Publication
(10) Pub. No.: US 2018/0036487 A1
(43) Pub. Date: Feb. 8, 2018

(54) CERAMIC FILTER FOR SYRINGE AND MANUFACTURING METHOD THEREFOR

(71) Applicant: SHINHAN CERAMIC CO., LTD., Siheung-si, Gyeonggi-do (KR)

(72) Inventors: Sung Ho KANG, Yongin-si Gyeonggi-do (KR); In Sub KIM, Yongin-si, Gyeonggi-do (KR); Jin Uk LEE, Goyang-si, Gyeonggi-do (KR); Chul Kyu SONG, Seoul (KR); Jin Oh YANG, Seoul (KR); Hyeon Min KANG, Chungju-si, Chungcheongbuk-do (KR); Soo Young SHIN, Incheon (KR)

(21) Appl. No.: 15/555,017

(22) PCT Filed: May 10, 2016
(86) PCT No.: PCT/KR2016/004856

§ 371 Date: Aug. 31, 2017

(30) Foreign Application Priority Data
May 14, 2015 (KR) 10-2015-0067606

(51) Int. Cl. A61M 5/31; C04B 35/628; C04B 35/64

(52) U.S. Cl. CPC A61M 5/3145; C04B 35/64; C04B 35/62813; A61M 5/165

(57) ABSTRACT

This invention relates to a ceramic filter for a syringe and a method of manufacturing the same, and more particularly to a ceramic filter for a syringe, which has filter performance equal to or higher than that of a metal filter and which includes alumina (Al₂O₃) and a sintering aid, thus solving various problems encountered while using the filter, and to a method of manufacturing the same.

### TECHNICAL FIELD

The present invention relates to a ceramic filter for a syringe and a method of manufacturing the same. More particularly, the present invention relates to a ceramic filter for a syringe, which includes alumina (Al₂O₃) and a sintering aid as raw materials to thus secure improved bioaffinity and chemical stability, and to a method of manufacturing the same.

### BACKGROUND ART

Generally, a syringe includes a cylinder, which is used for injecting a medicine into a patient after being filled with the medicine and which has a space formed so that an injection liquid is stored therein, a piston, which reciprocates in a chamber formed in the cylinder so as to suck or discharge the injection liquid, a needle holder, which is fitted to the outer circumferential side of a neck part formed at the front of the cylinder, and an injection needle, which is insert-molded at an end of the needle holder.

After the upper portion of an ampoule containing the injection liquid is broken to open the ampoule, the injection needle of the syringe having the above-described constitution is inserted into the opened portion of the ampoule, and the piston is then retracted, whereby the cylinder sucks the injection liquid in the ampoule.

When the injection needle is stuck into the affected part of the patient and the piston is then pushed, the injection liquid in the cylinder is injected into the patient through the injection needle.

However, with regard to the suction and addition of the injection liquid, when the upper portion of the ampoule is broken to open the ampoule, glass fragments are scattered and some of the glass fragments enter the inside of the ampoule. The glass fragments that enter the inside of the ampoule are transported into the syringe and mixed with the injection liquid when the syringe sucks the injection liquid, thus having a catastrophic adverse effect on the patient if the resultant liquid is added to the human body.

In order to solve this problem, a filter of U.S. Pat. No. 5,125,415 has been proposed.

When the injection liquid contained in the ampoule is injected into the syringe using a filter including porous polyethylene, the injection liquid may chemically react with the filter to thus oxidize the filter or to cause a material change of the injection liquid.

Meanwhile, a filter including an open-cell-type metal material is used in consideration of the problems with a conventional filter including a polymer material. However, the metal material filter has a problem in that oxidation occurs.

### DISCLOSURE

### Technical Problem

Accordingly, the present invention has been made keeping in mind the above problems occurring in the prior art, and an object of the present invention is to provide a ceramic filter for a syringe and a method of manufacturing the same. The ceramic filter includes alumina (Al₂O₃) and a sintering aid, which is not deformed, unlike a conventional filter including a polymer component and which prevents the problem of a metal filter that may be oxidized and consequently eluted.

### Technical Solution

In order to accomplish the above object, the present invention provides a method of manufacturing a ceramic filter for a syringe, the method including

(A) adding alumina (Al₂O₃) having a particle size of 3 to 120 μm, a sintering aid, a dispersant, and an antifoaming agent, and performing uniform primary dispersion and mixing in a wet manner to prepare a mixture;

(B) adding an organic additive, which includes a binder, a plasticizer, a release agent, and a humectant, to the mixture in a process (A), and performing secondary dispersion and mixing to bind raw materials;

(C) granulating the raw materials in a process (B) using a spray dryer device to form granules having a particle size of 20 to 200 μm;

(D) adding the granules obtained during a process (c) to a mold to perform powder pressing;

(E) multi-stage heat treating a molded substance obtained during a process (D);

(F) performing a barrel process for removing burs attached to the molded substance prepared during a process (E); and

(G) cleaning a product obtained during a process (F) using an ultrasonic wave, followed by drying.

The mixture in the process (A) includes 75 to 99 parts by weight of the alumina, 1 to 25 parts by weight of the sintering aid, 0.1 to 5 parts by weight of the dispersant, and 0.01 to 1 part by weight of the antifoaming agent.

The sintering aid is a single oxide or a mixture of two or more oxides selected from the group consisting of Al, Mg, Si, and Ca oxides.

The organic additive in the process (B) is added in a content of 50 to 250 parts by weight based on 100 parts by weight of the organic mixture in the process (A).

The organic additive in the process (B) includes 0.1 to 5 parts by weight of the dispersant, 0.01 to 1 parts by weight of the antifoaming agent, 1 to 10 parts by weight of the binder, 0.01 to 2 parts by weight of the plasticizer, 0.5 to 3 parts by weight of the release agent, and 0.01 to 1 parts by weight of the humectant.

The multi-stage heat treating of the process (E) includes a degreasing process for performing primary heat treatment at a low temperature ranging from room temperature to 800° C., thus removing the organic additive added during the process (A), and a sintering process for performing secondary heat treatment at a high temperature ranging from room temperature to 1600° C. after the degreasing process.

The present invention also provides a ceramic filter for a syringe manufactured using the above-described manufacturing methods, the ceramic filter including an open-pore structure having a porosity of 25 to 50%, a specific gravity of 3.0 to 4.5, and an open pore size of 2 to 100 μm.

### Advantageous Effects

The ceramic filter for a syringe manufactured using the manufacturing method provided in the present invention is not deformed, unlike a conventional filter including a polymer material, and is not oxidized, unlike a metal filter. The ceramic filter has a porosity of 25 to 50%, a pressure of 1.2 bar or less, a pore size of 2 to 100 μm, and excellent filter performance.

The present invention has the effect of improving the performance of a conventional filter for a syringe and improving bioaffinity and chemical stability, thereby solving problems occurring during use.

### DESCRIPTION OF DRAWINGS

FIG. 1 is an electron microscope photograph showing the surface of a ceramic filter manufactured using the manufacturing method of present invention;

FIG. 2 is a process flowchart showing the manufacturing method of the present invention;

FIG. 3 is an optical microscope photograph showing the surface of the ceramic filter manufactured according to the present invention; and

FIG. 4 is a digital photograph showing a syringe equipped with the ceramic filter manufactured using the manufacturing method of the present invention.

### BEST MODE

Hereinafter, the present invention will be described in more detail.

The present invention relates to a ceramic filter for a syringe and a method of manufacturing the same. More particularly, the present invention relates to a ceramic filter for a syringe, which has filter performance equal to or higher than that of a metal filter and which includes any one of alumina (Al₂O₃) or silicon carbide (SiC) and a sintering aid, thus solving various problems encountered while using the filter, and a method of manufacturing the same.

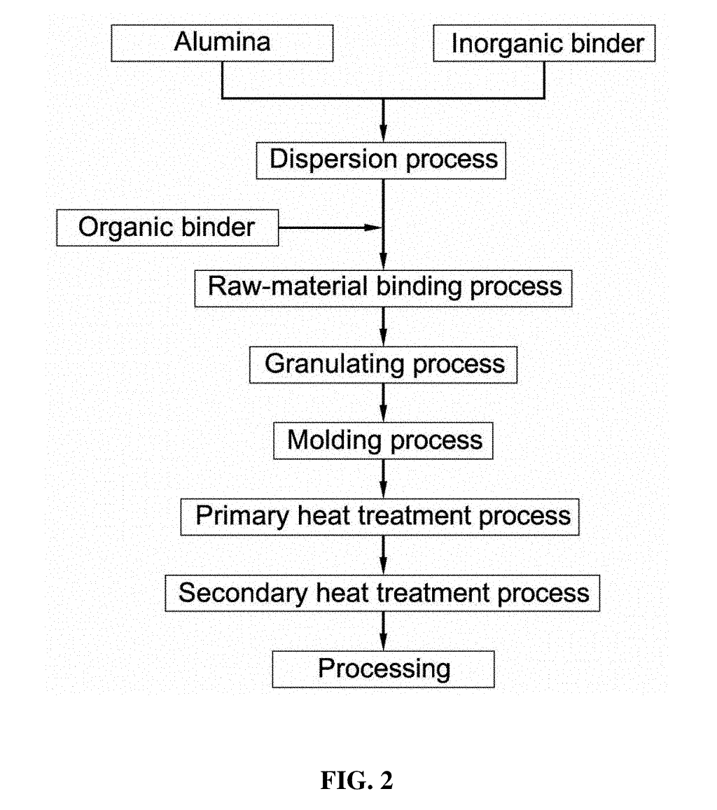

The method of manufacturing the ceramic filter for the syringe according to the present invention will be described using alumina (Al₂O₃) as an example, and as shown in FIG. 2, which is the accompanying drawing, the method includes:

(A) adding alumina (Al₂O₃) having a particle size of 3 to 120 μm, a sintering aid, a dispersant, and an antifoaming agent, and performing uniform primary dispersion and mixing in a wet manner to prepare a mixture;

(B) adding an organic additive, which includes a binder, a plasticizer, a release agent, and a humectant, to the mixture in a process (A), and performing secondary dispersion and mixing to bind raw materials;

(C) granulating the raw materials in a process (B) using a spray dryer device to form granules having a particle size of 20 to 200 μm;

(D) adding the granules obtained during a process (c) to a mold to perform powder pressing;

(E) multi-stage heat treating a molded substance obtained during a process (D);

(F) performing a barrel process for removing burs attached to the molded substance prepared during a process (E); and (G) cleaning a product obtained during a process (F) using ultrasonic waves, followed by drying.

According to the present invention, the mixture in the process (A) includes 75 to 99 parts by weight of the alumina, 1 to 25 parts by weight of the sintering aid, 0.1 to 5 parts by weight of the dispersant, and 0.01 to 1 part by weight of the antifoaming agent. When the content of the alumina falls outside of the threshold values, the filter performance is reduced, and when the content of the sintering aid falls outside of the threshold values, the mechanical strength may be reduced.

The dispersant to be used may be any one selected from the group consisting of polycarboxylate, sorbitan ester, polyether, amide, sodium polycarboxylate, ammonium polycarboxylate, condensed ammonium naphthalene sulfonate, alkylammonium, polyvalent alcohol ester, and a non-ionic surfactant. It is more preferable to use polycarboxylate, sorbitan ester, polyether, amide, sodium polycarboxylate, or ammonium polycarboxylate.

Further, the antifoaming agent may be any one selected from the group consisting of alcohols, polyethers, metal soaps, and amides, and it is more preferable to use polyethers.

Further, when the content of the dispersant is less than 0.1 parts by weight, the dispersion may not be performed well, which causes the formation of macropores. When the content is more than 5 parts by weight, the time required for a debinding process is increased and the strength of a product is reduced. When the content of the antifoaming agent falls outside of the threshold values, it is difficult to obtain a satisfactory antifoaming effect.

According to the present invention, the content of the sintering aid is 1 to 20 parts by weight, and the sintering aid is a single oxide or a mixture of two or more oxides selected from the group consisting of Al, Mg, Si, and Ca oxides.

When the content of the sintering aid is less than part by weight, the strength of the sintered body is reduced, and when the content is more than 20 parts by weight, the filter performance is reduced.

According to the present invention, the organic additive is preferably added in a content of 50 to 250 parts by weight based on 100 parts by weight of the mixture in the process (A). When the content is less than 50 parts by weight, the dispersing and molding properties are deteriorated, which causes the occurrence of burs. When the content is more than 250 parts by weight, the time required for a degreasing process is increased and macropores are formed in the ceramic filter microstructure, thus deteriorating the filter performance.

Preferably, the organic additive in the process (B) includes 0.01 to 1 parts by weight of the antifoaming agent, 1 to 10 parts by weight of the binder, 0.01 to 2 parts by weight of the plasticizer, 0.5 to 3 parts by weight of the release agent, and 0.01 to 1 parts by weight of the humectant.

According to the present invention, the heat-treating process of the process (E) is a multi-stage heat-treating process. The heat-treating process includes a degreasing process for performing primary heat treatment at a low temperature range of room temperature to 800° C., thus removing the organic binder remaining after the molding process, and a sintering process for performing secondary heat treatment at a high temperature range of up to 1600° C. after the degreasing process.

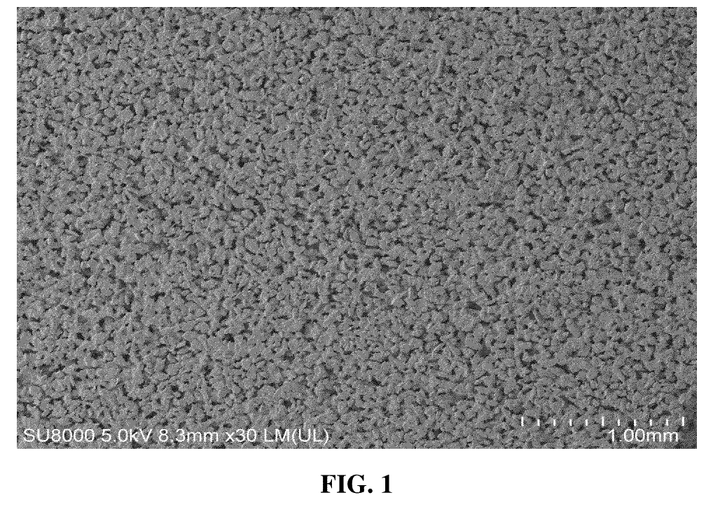

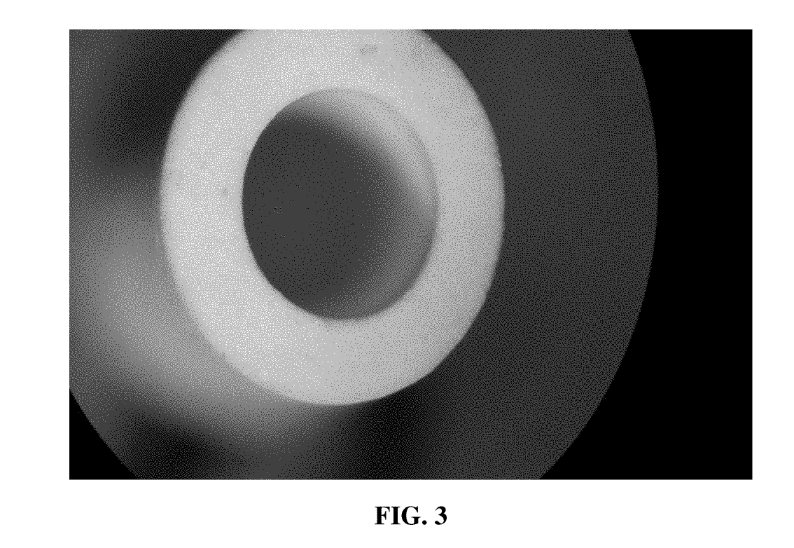

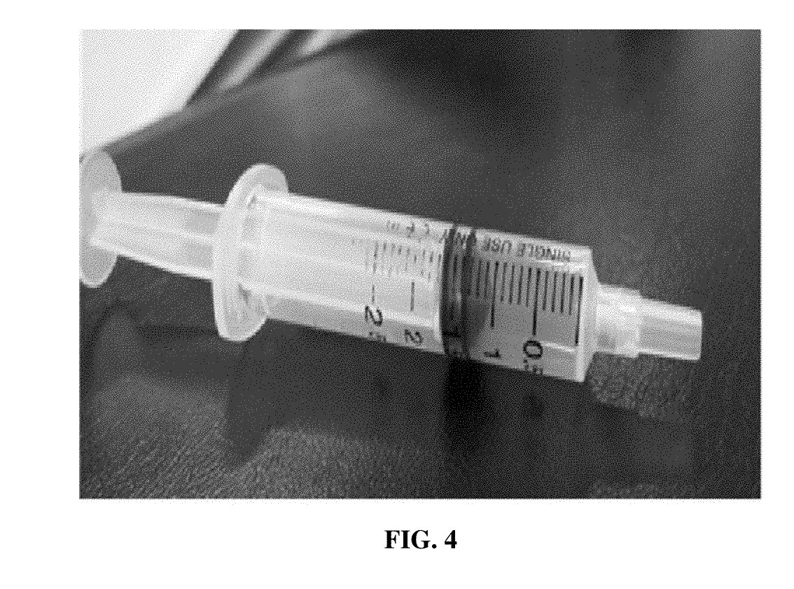

According to the manufacturing method of the present invention thus constituted, as shown in FIGS. 1 to 4, which are the accompanying drawings, there may be provided a ceramic filter for a syringe, which includes an open-pore structure having a porosity of 25 to 50%, a specific gravity of 3.0 to 4.5, and an open pore size of 2 to 100 μm.

### MODE FOR INVENTION

Hereinafter, the present invention will be described in more detail with reference to preferred embodiments. However, the present invention is not limited to the following Examples which are set forth for the purpose of illustration, but can be modified in various ways within the scope of the present invention.

### Examples 1 to 3

<Preparation of Materials>

1. Alumina and silicon carbide: Alumina and silicon carbide having a particle size of 3 to 120 μm were prepared.

2. Sintering aid: 1 to 20 parts by weight of a sintering aid was prepared.

3. Dispersant: Polycarboxylate

4. Antifoaming agent: Dimethylsilicon

5. Organic additive: The organic additive was prepared so as to include 0.1 to 5 parts by weight of a dispersant, 0.01 to 1 parts by weight of an antifoaming agent, 1 to 10 parts by weight of a binder, 0.01 to 2 parts by weight of a plasticizer, 0.5 to 3 parts by weight of a release agent, and 0.01 to 1 parts by weight of a humectant mixed therein.

<Manufacturing Process>

The alumina, the sintering aid, the dispersant, and the antifoaming agent were primarily dispersed and mixed in a wet manner according to the composition ratios (unit: parts by weight) described in the following Tables 1 and 2. After the primary dispersion and mixing were finished, the organic additive was added to perform secondary dispersion and mixing, and the raw materials were bound. After the binding of the raw materials was finished, the raw materials were granulated using a spray-dryer device to form granules having a particle size of 20 to 200 μm. The obtained granules were added to a mold and subjected to powder pressing, thus forming a crucible shape.

The molded substance was subjected to primary heat treatment at a low temperature ranging from room temperature to 800° C. to remove the organic binder remaining after the molding process, and was then subjected to secondary heat treatment at a high temperature range of 1600° C. to perform a sintering process.

A barrel process was performed to remove burs attached to the sintered molded substance, and the obtained product was cleaned using an ultrasonic wave and then dried, thus preparing a filter prototype.

The physical properties of the prepared prototype were measured, and the results are set forth in the following Tables 3 and 4.

TABLE 1

| | | Sintering | Dis- | Antifoaming | Organic |
|---|---|---|---|---|---|
| Classification | Alumina | aid | persant | agent | additive |
| Example 1 | 75 | 1 | 0.1 | 0.01 | 0.05 |
| Example 2 | 85 | 15 | 3 | 0.05 | 4.5 |
| Example 3 | 99 | 25 | 5 | 1 | 15 |

TABLE 2

| | Silicon | Sintering | | Antifoaming | Organic |
|---|---|---|---|---|---|
| Classification | carbide | aid | Dispersant | agent | additive |
| Example 1 | 60 | 1 | 0.1 | 0.02 | 0.05 |
| Example 2 | 80 | 10 | 2 | 0.07 | 4 |
| Example 3 | 98 | 25 | 4 | 1 | 13 |

TABLE 3

| | Porosity | Specific | Open pore | Deformation | |
|---|---|---|---|---|---|
| Classification | (%) | gravity | size (μm) | rate (%) | Elution |
| Example 1 | 25 | 3 | 2 | 0 | 0 |
| Example 2 | 35 | 3.7 | 10 | 0 | 0 |
| Example 3 | 50 | 4.5 | 100 | 0 | 0 |

TABLE 4

| | Porosity | Specific | Open pore | Deformation | |
|---|---|---|---|---|---|
| Classification | (%) | gravity | size (μm) | rate (%) | Elution |
| Example 1 | 25 | 3 | 2 | 0 | 0 |
| Example 2 | 40 | 3.8 | 40 | 0 | 0 |
| Example 3 | 50 | 4.5 | 90 | 0 | 0 |

⊚ Porosity: Test Method—KSF 2527

Examples 1 to 3 exhibited a change in porosity depending on the content of the alumina, the inorganic binder, the dispersant, the antifoaming agent, and the organic additive (ref: Tables 1 and 2). In Example 1, the porosity was 25% due to the low content of the alumina and the inorganic binder, and in Example 3, the porosity was 50% due to the excessive content of the alumina. Preferably, it is judged that Example 2 provides optimum raw-material mixing and processing conditions.

⊚ Specific gravity: Test Method—ASTMD 792

Examples 1 to 3 exhibited a change in specific gravity depending on the content of the alumina, the inorganic binder, the dispersant, the antifoaming agent, and the organic additive (ref: Tables 1 and 2). In Example 1, the specific gravity was 3.5 due to the low content of the alumina, and in Example 3, the porosity was 3.7 due to the excessive content of the alumina. Preferably, it is judged that Example 2 provides optimum raw-material mixing and processing conditions.

⊚ Open pore size: Test Method—ISO 2738

Examples 1 to 3 exhibited a change in open pore size depending on the content of the alumina, the inorganic binder, the dispersant, the antifoaming agent, and the organic additive (ref: Tables 1 and 2). In Example 1, the open pore size was 2 due to the low content of the inorganic binder depending on the content of the alumina, and in Example 3, the pore size was 100 μm due to the excessive content of alumina. Preferably, it is judged that Example 2 provides optimum raw-material mixing and processing conditions.

⊚ Deformation rate: Test Method—ASTMD 638

A ceramic filter for a syringe was manufactured according to the raw-material mixing process of Examples 1 to (ref: Tables 1 and 2). The deformation rate owing to shrinkage and expansion of polymer and metal filters according to the injection pressure of medicines was not exhibited, and this is considered to be a merit of the ceramic filter.

⊚ Elution: Test Method—KSK 1204

A ceramic filter for a syringe was manufactured according to the raw-material mixing process of Examples 1 to (ref: Tables 1 and 2). The elution owing to oxidation and chemical reactions of polymer and metal filters according to the injection of medicines was not exhibited, and this is considered to be a merit of the ceramic filter.

The ceramic filter for the syringe has excellent filter performance, and is not deformed and not eluted. The ceramic filter includes alumina and an inorganic binder as raw materials to thus secure improved bioaffinity and chemical stability.

1. A method of manufacturing a ceramic filter for a syringe, the method comprising:
(A) adding alumina (Al₂O₃) having a particle size of 3 to 120 μm, a sintering aid, a dispersant, and an antifoaming agent, and performing uniform primary dispersion and mixing in a wet manner using a ball mill to prepare a mixture;
(B) adding an organic additive, which includes a binder, a plasticizer, a release agent, and a humectant, to the mixture in a process (A), and performing secondary dispersion and mixing to bind raw materials;
(C) granulating the raw materials in a process (B) using a spray dryer device to form granules having a particle size of 20 to 200 μm;
(D) adding the granules obtained during a process (c) to a mold to perform powder pressing;
(E) multi-stage heat treating a molded substance obtained during a process (D);
(F) performing a barrel process for removing a bur attached to the molded substance prepared during a process (E); and
(G) cleaning a product obtained during a process (F) using an ultrasonic wave, followed by drying.

2. The method of claim 1, wherein the mixture in the process (A) includes 75 to 99 parts by weight of the alumina, 1 to 25 parts by weight of the sintering aid, 0.1 to 5 parts by weight of the dispersant, and 0.01 to 1 parts by weight of the antifoaming agent.

3. The method of claim 1, wherein the sintering aid in the process (A) is included in a content of 1 to 20 parts by weight.

4. The method of claim 3, wherein the sintering aid is a single oxide or a mixture of two or more oxides selected from the group consisting of Al, Mg, Si, and Ca oxides.

5. The method of claim 1, wherein the organic additive in the process (B) is added in a content of 50 to 250 parts by weight based on 100 parts by weight of the mixture in the process (A).

6. The method of claim 1, wherein the organic additive in the process (B) includes 0.01 to 1 parts by weight of the antifoaming agent, 1 to 10 parts by weight of the binder, 0.01 to 2 parts by weight of the plasticizer, 0.5 to 3 parts by weight of the release agent, and 0.01 to 1 parts by weight of the humectant.

7. The method of claim 1, wherein the multi-stage heat treating of the process (E) includes a degreasing process for performing primary heat treatment at a low temperature ranging from room temperature to 800° C., thus removing the organic additive added during the process (A); and a sintering process for performing secondary heat treatment at a high temperature ranging from up to 1600° C. after the degreasing process.

8. The method of claim 1, wherein the alumina is silicon carbide (SiC).

9. A ceramic filter for a syringe manufactured using the manufacturing method of claim 1, the ceramic filter comprising:
an open-pore structure having a porosity of 25 to 50%, a specific gravity of 3.0 to 4.5, and an open pore size of 2 to 100 μm.
